(19) United States
(12) Patent Application Publication
(10) Pub. No.: US 2015/0313601 A1
(43) Pub. Date: Nov. 5, 2015

(54) MINIMALLY INVASIVE SURGICAL CLIP APPLIER

(71) Applicant: VITALITEC INTERNATIONAL, INC., Plymouth, MA (US)

(72) Inventors: David A. Walsh, Reading, MA (US); Wayne Robert Knupp, JR., Kingston, MA (US); Robert Swierczek, Salem, MA (US)

(73) Assignee: VITALITEC INTERNATIONAL, INC., Plymouth, MA (US)

(21) Appl. No.: 14/697,806

(22) Filed: Apr. 28, 2015

Related U.S. Application Data
(60) Provisional application No. 61/986,281, filed on Apr. 30, 2014.

(51) Int. Cl. A61B 17/128

(52) U.S. Cl. CPC A61B 17/1285; A61B 2017/00367

(57) ABSTRACT

A surgical clip applier having an adjustable jaw open dimension, including an instrument body having jaws at a distal end and an actuator at a proximal end; an actuator shaft movable longitudinally relative to the instrument body to transmit actuating force from the actuator to the jaws; and an adjusting member threadedly engaged with the actuator shaft and configured to engage the instrument body at a jaw open position, wherein rotation of the adjusting member relative to the actuating shaft moves the adjusting member longitudinally relative to the actuating shaft to adjust a jaw open position of the jaws when the adjusting member contacts the instrument body.

### BACKGROUND OF THE INVENTION

The invention relates to a surgical clip applier and, more particularly, to a surgical clip applier for use in minimally invasive procedures and having an adjusting mechanism for adjusting the dimension of the jaws of the applier in the open dimension.

The open position of a clip applier, especially a clip applier for minimally invasive surgical (MIS) procedures, is a critical aspect of the device. When the jaws open, if they open too much, a clip may not be firmly held between the jaws and could fall out of the appliers into the surgical site. On the other hand, if the jaws do not open wide enough, then clip loading can be difficult or impossible, and the clip may not load correctly, which could also lead to problems with placement and/or application at the surgical site.

Based upon the above, it is clear that the need exists for an improved structure for more reliably setting the correct open position of an MIS clip applier.

### SUMMARY OF THE INVENTION

In accordance with the present invention, an MIS clip applier is provided which has an adjustment mechanism for producing the correct open dimension in a jaw open position thereby ensuring that the open dimension of the clip applier will securely hold clips without being too tight. This in turn allows for much more reliable placement of clips during MIS procedures. Further, the adjustment mechanism remains adjustable throughout the life of the instrument, which is particularly useful in addressing any wear or mistreatment to the instrument during the course of its use.

In accordance with a further feature of the present invention, the adjustable mechanism is designed such that a non-standard tool is required to make any adjustments, and in this way the adjustment mechanism is prevented from being altered by an unskilled or unintended operator, and the adjustment mechanism is also adapted to prevent unintentional migration.

The surgical clip applier in accordance with the present invention includes an instrument body having jaws at a distal end and an actuator at a proximal end; an actuator shaft movable longitudinally relative to the instrument body to transmit actuating force from the actuator to the jaws; and an adjusting member threadedly engaged with the actuator shaft and configured to engage the instrument body at a jaw open position, wherein rotation of the adjusting member relative to the actuating shaft moves the adjusting member longitudinally relative to the actuating shaft to adjust a jaw open position of the jaws when the adjusting member contacts the instrument body.

A clear advantage of the inventive design is that it fully addresses the challenge of getting the correct jaw opening dimension, which is a very complicated procedure due to the tolerance stack up in all the components in the actuating mechanism. In other words, since the jaw opening and closing mechanism utilizes a series of components, and each component will have mechanical tolerance, the proper full open position of the device is complicated due to each of the mechanical tolerances of each of these components. With the present adjustment mechanism, the inventive MIS clip applier can be precisely adjusted despite such tolerances to the proper open dimension.

### BRIEF DESCRIPTION OF THE DRAWINGS

A detailed description of preferred embodiments of the present invention follows with reference to the attached drawings, wherein:

FIG. 1 is a sectional view of a surgical clip applier in accordance with the present invention in an open jaw position taken along the lines A-A of FIG. 2;

FIG. 2 is a side view of a surgical clip applier in accordance with the present invention in the open jaw position;

FIG. 3 is a sectional view of a surgical clip applier in accordance with the present invention in a closed jaw position and taken along the lines of B-B of FIG. 4;

FIG. 4 is a side view of a surgical clip applier in accordance with the present invention in the closed jaw position;

FIG. 5 is an enlarged view of the proximal components of the adjustment mechanism of a surgical clip applier in accordance with the present invention, in the open jaw position;

FIG. 6 is an enlarged view of the proximal components of the adjustment mechanism of a surgical clip applier in accordance with the present invention, in the closed jaw position;

FIGS. 7-9 further illustrate an embodiment of an adjustment knob in accordance with the present invention; and

FIG. 10 illustrates an embodiment of an adjustment tool in accordance with the present invention.

### DETAILED DESCRIPTION

The invention relates to a surgical clip applier, especially a surgical clip applier designed for use in minimally invasive surgery (MIS) wherein the clip applier is to be positioned to a surgical site through a laparoscopic cannula.

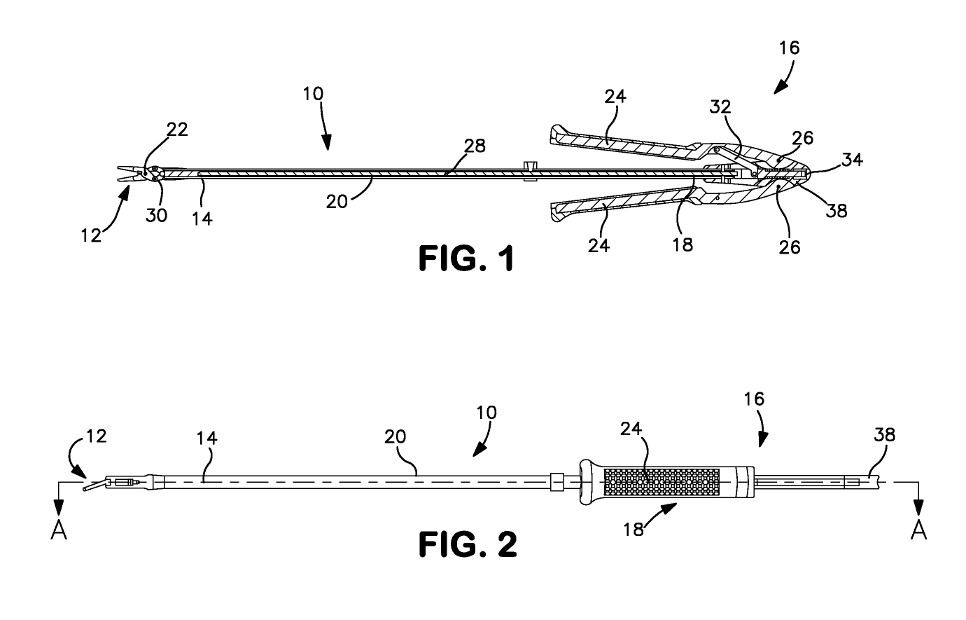

FIG. 1 shows a surgical clip applier 10 in accordance with the present invention. Applier 10 has jaws 12 at a distal end 14 and an actuator assembly 16 at a proximal end 18. An instrument body 20 extends between proximal end 18 and distal end 14 and is advantageously an elongate, hollow and substantially rigid structure. Jaws 12 are pivotally mounted at distal end 14, for example around a pivot pin 22.

Actuator assembly 16 in the illustrated embodiment has two levers 24 which are pivotally mounted relative to body 20, for example at pivot points 26.

An actuator shaft 28 is slidably positioned within body 20 and extends from actuator assembly 16 to jaws 12. Shaft 28 is connected to jaws 12, preferably through links 30 pivotally connected through jaws 12, such that longitudinal translation of shaft 28 within body 20 opens and closes the jaws.

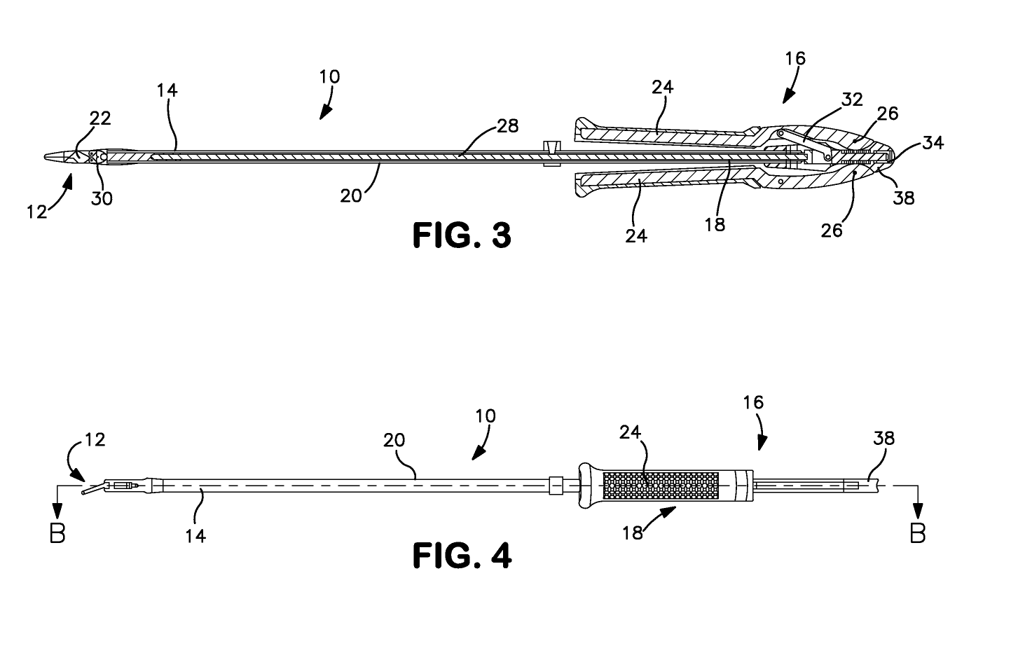

At proximal end 18, shaft 28 is operatively associated with levers 24, in this embodiment through a link 32. Link 32 is pivotally connected to one lever 24 at one end, and to shaft 28 at the other end. As will be further discussed below, opening and closing of levers 24 relative to body 20 moves link 32 and, thereby, actuator shaft 28, as desired. A closing of levers 24 moves shaft 28 proximally relative to body 20, which positions jaws 12 into the closed position. (See FIGS. 3 and 4). Opening of levers 24 relative to body 20 causes a distal movement of actuator shaft 28 relative to body 20, thereby causing an opening of jaws 12 (FIGS. 1 and 2).

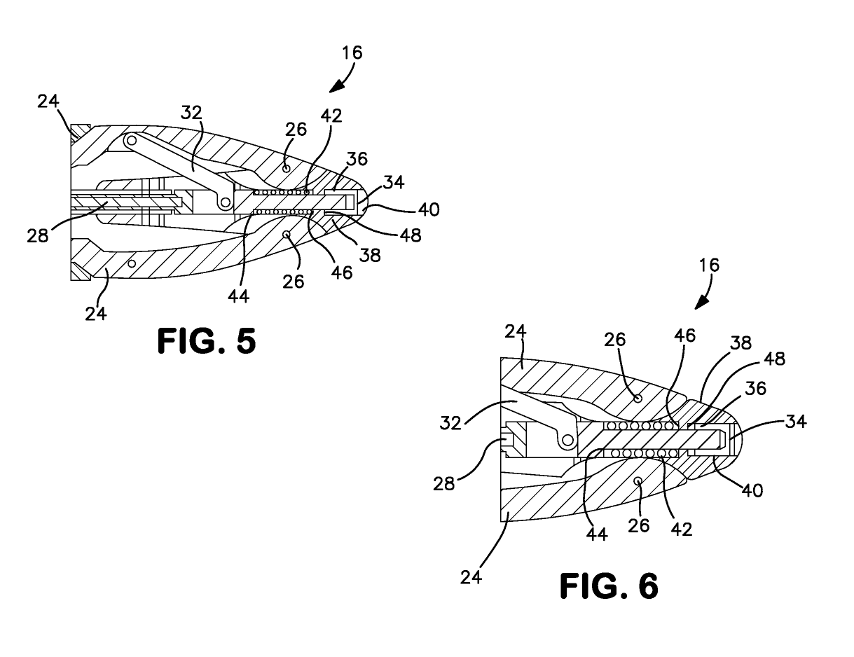

Referring also to FIGS. 5 and 6, the adjustment assembly in accordance with the present invention is further illustrated, and this assembly acts to create an adjustably-positioned stop position such that when levers 24 are opened relative to body 20, jaws 12 will open to the correct dimension.

The adjusting assembly in accordance with the present invention is preferably a threaded adjustment knob 34 which is threadedly engaged with a proximal end of actuator shaft 28. Adjustment knob 34 has additional structure, in this case a distally extending sleeve 36 which extends radially outwardly from shaft 28, to engage against body 20 in the fully open position (FIG. 5). As shown in FIG. 5, body 20 can include a proximally positioned end body 38 having an internal bore for accommodating a proximal end of shaft 28 and adjustment knob 34. As shown in FIG. 5, end body 38 also has rounded outer surfaces for pivotally receiving levers 24 at pivot points 26.

From a consideration of FIGS. 2, 4 and 5-6, it should be appreciated that end body 38 creates a recessed area for receiving adjustment knob 34 such that adjustment knob 34 is shielded from unintentional contact or adjustment.

It should be appreciated that adjustment knob 34 is rotated so as to change position of knob 34 relative to shaft 28 in the course of adjusting the desired exact position of knob 34 to produce the proper open dimension in the jaw open position. Thus, it is necessary to rotate adjustment knob 34 during adjustment of the device. In accordance with a further aspect of the present invention, and in order to prevent unauthorized or unskilled adjustment of adjustment knob 34, knob 34 is advantageously provided with a non-standard engagement structure. In other words, knob 34 is provided with structure for engaging with a tool, but which is preferably not a typical screwdriver or Allen wrench type engagement structure. Further, a corresponding or matching tool adapted for engagement with knob 34 is advantageously provided with applier 10 so that skilled and intended persons can adjust knob 34 as desired.

In further accordance with this aspect of the invention it should again be appreciated that recessed bore 40 of end body 38 is sufficiently deep that knob 34 is completely received within recessed bore 40, preferably with no portion of knob 34 extending beyond end body 38. Further, and as best illustrated in FIGS. 2 and 4, end body 38 can also have a proximally concave structure to further assist in preventing any unintended contact with adjustment knob 34.

Still referring to FIGS. 5 and 6, a spring 42 can be positioned within end body 38 such that it exerts a biasing force in a distal direction against a shoulder 44 of shaft 28, and in a proximal direction against a shoulder or other structure 46 of end body 38. In this manner, spring 42 serves to bias shaft 28 in a distal direction and keep knob 34 engaging against stop surface 48 of end body 38. In this way, when levers 24 are released or opened, spring 42 ensures that knob 34 through shaft 28 positioned jaws 12 into the desired and exactly adjusted open dimension.

As shown in FIG. 6, when levers 24 are compressed to close jaws 12, shaft 28 moves proximally along with knob 34 which moves proximally a small distance away from stop surface 48. When levers 24 are released, these components move back to the position of FIG. 5.

In accordance with a further aspect of the present invention, adjustment knob 34 is preferably axially slotted to produce a spring effect on the threaded engagement with the actuator shaft 28. This helps to prevent knob 34 from migrating unintentionally relative to shaft 28 and, thereby, altering the fine adjustment of applier 10. This mechanism operates in a similar manner to a standard lock washer, wherein the slot causes the material to act like a spring and maintain constant pressure and friction between knob 34 and shaft 28, which helps to prevent any relative movement of these components.

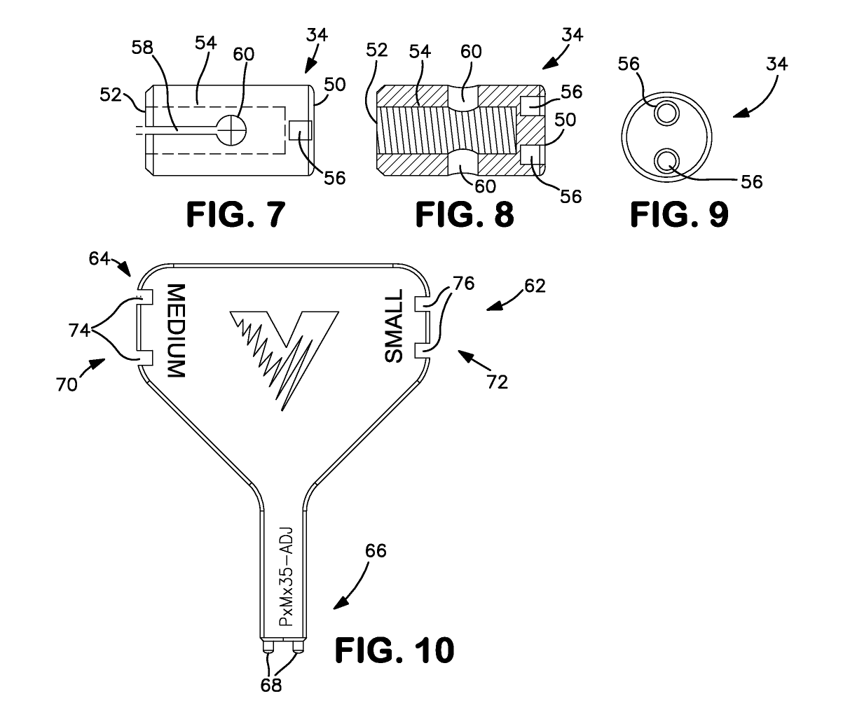

FIGS. 7-9 illustrate further features of an adjustment knob 34 in accordance with the present invention. As shown, adjustment knob 34 has a proximal end 50 and a distal end 52, as well as a threaded internal surface 54 for engaging with shaft 28. As shown in these drawings, proximal end 50 has non-standard structure for engaging with a tool, and this structure, in this embodiment, is provided in the form of two spaced holes or sockets 56. Sockets 56 are engaged with an adjustment tool as will be discussed further below when it is needed to rotate adjustment knob 34 relative to shaft 28 and thereby adjust the axial position of knob 34 relative to shaft 28.

Still referring to FIG. 7-9, adjustment knob 34 can advantageously have a slot 58 through the sidewall of knob 34, preferably extending from a circular opening 60 to distal end 52 as shown. This slotted structure, coupled with sizing of internal thread 54 to have a compression fit on the threads of shaft 28, serves to provide a spring interaction between knob 34 and shaft 28 to help prevent unintended rotation of knob 34 relative to shaft 28. FIGS. 7 and 8 are shown rotated 90 degrees from each other around the longitudinal axis of knob 34 and have dashed lines to better illustrate the internal structure of slot 58, openings 60 and sockets 56.

FIG. 9 is an end view of knob 34 and further illustrates the two spaced sockets 56 which are used to engage knob 34 with a specialized adjustment tool as will be discussed below.

FIG. 10 shows an adjustment tool 62 which is designed specifically for use with knob 34 when knob 34 is to be rotated to adjust its position and, thereby, the width of the opening of jaws in a full open position.

As shown, tool 62 can have a handle portion 64 and a tool portion 66. Tool portion 66 preferably has two extending prongs 68 which are sized and spaced to match with sockets 56 of knob 34. Thus, when tool 62 is to be used to engage knob 34, prongs 68 are inserted into sockets 56 to allow secure engagement of tool 62 with knob 34 and appropriate rotation of same.

Tool 62 can also be provided with one or more gap setting structures as shown at 70 and 72. These structures 70, 72 can be provided as two spaced indentations 74, 76, which are sized to accommodate the tips of the jaws of the applier. Indentations 74, 76 are spaced on tool 62 at the correct spacing of jaws for particular sizes of clips. In the embodiment shown in FIG. 10, indentations 74 are set to the proper width for a medium clip, and indentations 76 are set to the proper width of a small clip. Tool 62 also has indicia in close proximity to close proximity to indentations 74, 76 to convey this information to the user of tool 62. It should be appreciated that tool 62 having different spaced indentations 74, 76 advantageously serves to allow tool 62 to easily adjust a clip applier to both sizes of clips.

In use, tool 62 could be used to position jaws at the proper width by matching the jaws to indentations 74, 76 as desired, and then engaging tool 62 with knob 34 to rotate the knob into contact with surface 48 of end body 38. Once knob 34 is appropriately positioned, indentations 74, 76 can then be again matched to the jaws of the device in the full open position to confirm that the applier is properly adjusted.

It should be appreciated that while, in this embodiment, the sockets and prongs of knob 34 and tool 62 are one embodiment of a non-standard tool for use in providing authorized adjustment of the applier, the specific non-standard structures could be provided in different ways, with different shapes and numbers of sockets and prongs, and/or with prongs and sockets on the opposite member as well. Nevertheless, it is preferred to have the sockets be in knob 34 as this helps keep a low profile of knob 34 within the applier, and thereby helps to reduce the possibility of inadvertent contact and movement of the knob.

It is to be understood that the invention is not limited to the illustrations described and shown herein, which are deemed to be merely illustrative of the best modes of carrying out the invention, and which are susceptible to modification of form, size, arrangement of parts and details of operation. The invention, rather, is intended to encompass all such modifications which are within its spirit and scope as defined by the claims.

What is claimed is:

1. A surgical clip applier having an adjustable jaw open dimension, comprising:
an instrument body having jaws at a distal end and an actuator at a proximal end;
an actuator shaft movable longitudinally relative to the instrument body to transmit actuating force from the actuator to the jaws; and
an adjusting member threadedly engaged with the actuator shaft and configured to engage the instrument body at a jaw open position, wherein rotation of the adjusting member relative to the actuating shaft moves the adjusting member longitudinally relative to the actuating shaft to adjust a jaw open position of the jaws when the adjusting member contacts the instrument body.

2. The apparatus of claim 1, wherein the instrument body is an elongate member dimensioned for minimally invasive procedures.

3. The apparatus of claim 1, wherein the instrument body further comprises an end body having a recessed bore for receiving the adjusting member.

4. The apparatus of claim 3, wherein the actuator comprises at least one lever pivotably mounted to the end body and having a ink connected from the at least one lever to the actuator shaft whereby pivot of the at least one lever moves the actuator shaft longitudinally relative to the instrument body.

5. The apparatus of claim 1, further comprising a spring positioned between the shaft and the instrument body to bias the adjusting member into contact with the instrument body.

6. The apparatus of claim 1, wherein the adjustable member is recessed into the instrument body and accessible for adjustment of the jaw open dimension.

7. The apparatus of claim 1, wherein the adjustable member has a non-standard engagement structure, and further comprising a non-standard adjusting tool matched to the non-standard engagement structure.

8. The apparatus of claim 1, wherein the adjusting member is axially slotted to produce a spring effect with the actuator shaft to prevent unintentional movement of the adjusting member relative to the actuating shaft.
